(19) United States
(12) Patent Application Publication
(10) Pub. No.: US 2023/0270376 A1
(43) Pub. Date: Aug. 31, 2023

(54) EVALUATING THE STABILITY OF A JOINT IN THE FOOT AND ANKLE COMPLEX VIA WEIGHT-BEARING MEDICAL IMAGING

(71) Applicant: THE GENERAL HOSPITAL CORPORATION, Boston, MA (US)

(72) Inventors: Kartik Mangudi Varadarajan, Acton, MA (US); Alireza Borjali, Somerville, MA (US); Soheil Ashkani Esfahani, Cambridge, MA (US); Bart Lubberts, Cambridge, MA (US); Gregory R. Waryasz, Boston, MA (US); Christopher W. Digiovanni, Boston |, MA (US); Orhun K. Muratoglu, Cambridge, MA (US); Daniel Guss, Brookline, MA (US)

(21) Appl. No.: 18/015,023

(22) PCT Filed: Jul. 7, 2021
(86) PCT No.: PCT/US2021/040750

§ 371 Date: Jan. 6, 2023

Related U.S. Application Data
(60) Provisional application No. 63/048,827, filed on Jul. 7, 2020.

(51) Int. Cl. A61B 5/00; A61B 5/055; A61B 6/00; G06T 7/00; G16H 50/20; G16H 30/40; G06N 5/022

(52) U.S. Cl. CPC A61B 5/4595; A61B 5/055; A61B 5/7267; A61B 6/505; A61B 6/5217; G06N 5/022; G06T 7/0012; G16H 30/40; G16H 50/20; G06T 2207/10081; G06T 2207/10088; G06T 2207/20081; G06T 2207/20084; G06T 2207/30008

(57) ABSTRACT

Systems and methods are provided for evaluating a stability of a joint within the foot and ankle complex of a subject. The subject is instructed to assume a position in which the joint is bearing weight, and a three-dimensional medical image of the joint comprising a sequence of two-dimensional image slices is captured at a scanner. The sequence of two-dimensional image slices is provided to a predictive model, comprising an artificial neural network having at least one convolutional layer. A clinical parameter representing the stability of the joint at the predictive model is determined from at least the sequence of two-dimensional image slices.

### RELATED APPLICATIONS

This application claims priority from U.S. Provisional Application No. 63/048,827, filed 7 Jul. 2020 and entitled “EVALUATING THE STABILITY OF A JOINT IN THE FOOT AND ANKLE COMPLEX VIA WEIGHT-BEARING MEDICAL IMAGING,” the subject matter of which is hereby incorporated by reference in its entirety.

### TECHNICAL FIELD

This invention relates to medical imaging, and more specifically, to evaluation of the stability of a joint in the foot and ankle complex via weight-bearing medical imaging.

### BACKGROUND

Deep learning is part of a broader family of machine learning methods based on artificial neural networks with representation learning. Deep learning techniques can be supervised, semi-supervised, or unsupervised. The adjective “deep” in deep learning refers to the use of multiple layers in the network. Deep learning is a modern variation of neural networks which is concerned with an unbounded number of layers of bounded size, which permits practical application and optimized implementation, while retaining theoretical universality under mild conditions. In deep learning the layers are also permitted to be heterogeneous and to deviate widely from biologically informed connectionist models, for the sake of efficiency, trainability, and understandability.

### SUMMARY

In one example, a method is provided for evaluating a stability of a joint within the foot and ankle complex of a subject. The subject is instructed to assume a position in which the joint is bearing weight, and a three-dimensional medical image of the joint comprising a sequence of two-dimensional image slices is captured at a scanner. The sequence of two-dimensional image slices is provided to a predictive model, comprising an artificial neural network having at least one convolutional layer. A clinical parameter representing the stability of the joint at the predictive model is determined from at least the sequence of two-dimensional image slices.

In another example, a system includes a medical scanner configured to capture images of a joint within the foot and ankle complex of a subject while the joint is bearing a weight of the subject to provide a sequence of at least three medical images. A non-transitory computer readable medium stores instructions executable by a processor to provide a scanner interface that receives the sequence of at least three medical images from the medical scanner and a predictive model, comprising an artificial neural network having at least one convolutional layer, that determines a clinical parameter representing a stability of the joint from at least the set of at least two difference images.

In a further example, a method is provided for evaluating a stability of a joint within the foot and ankle complex of a subject. A subject is instructed to assume a position in which the joint is bearing weight, and a three-dimensional medical image of the joint comprising a sequence of two-dimensional image slices is captured at a scanner. The sequence of two-dimensional image slices is provided to a predictive model, comprising a plurality of convolutional neural networks and an arbitrator that receives the outputs of the plurality of convolutional neural networks. A clinical parameter representing the stability of the joint is determined at the arbitrator from the outputs of the plurality of convolutional neural networks.

### BRIEF DESCRIPTION OF THE DRAWINGS

FIG. 1 illustrates an example of a system for evaluating of the stability of a joint in the foot and ankle complex via weight-bearing medical imaging;

FIG. 2 illustrates a predictive model that can be used in a system for determining a clinical parameter via evaluation of sequential medical images;

FIG. 3 is a diagram illustrating a portion of the foot and ankle complex that contains the syndesmosis joint;

FIG. 4 illustrates one example of a method for evaluating of the stability of a joint in the foot and ankle complex via weight-bearing medical imaging;

FIG. 5 illustrates another example of a method for evaluating of the stability of a joint in the foot and ankle complex via weight-bearing medical imaging; and

FIG. 6 is a schematic block diagram illustrating an exemplary system of hardware components.

### DETAILED DESCRIPTION

A “clinical parameter,” as used herein, is any continuous, ordinal, or categorical parameter that represents a current or predicted future medical condition of a patient, and can include any value representing diagnosis of disease or injury or predicting a patient outcome.

As used herein, a “predictive model” is a mathematical model that either predicts a future state of a parameter or estimates a current state of a parameter that cannot be directly measured or for which direct measurement is impractical.

A “convolutional neural network,” as used herein, is any artificial neural network having at least one convolutional layer.

The “foot and ankle complex,” as used herein, refers to the mechanical system formed by the foot and the ankle, including the bones, joints, ligaments, and muscles within the foot and ankle. Joints within the foot and ankle complex can include fibrous joints, such as the syndesmosis joint, cartilaginous joints, and synovial joints within the foot and ankle.

Two machine learning models are “independent” when they do not directly or indirectly influence each other’s output.

As used herein, the term “subject” can refer to any warm-blooded organism including, but not limited to, a human being, a pig, a rat, a mouse, a dog, a cat, a goat, a sheep, a horse, a monkey, an ape, a rabbit, a cow, etc. The terms “patient” and “subject” can be used interchangeably herein.

Systems and methods provided herein utilize a deep learning model for analysis of sequential slices from weight-bearing medical images in three-dimensional medical images. This can include, for example, images generated via magnetic resonance imaging (MRI) and computed tomography (CT) scans, which consist of a stack or series of image slices. The proposed deep learning model includes an artificial neural network that analyzes the two-dimensional slices from the three-dimensional medical images to produce a parameter representing the stability of the joint. The use of this system can detect subtle signs of joint instability that could be missed by a technician or physician reviewing the medical image.

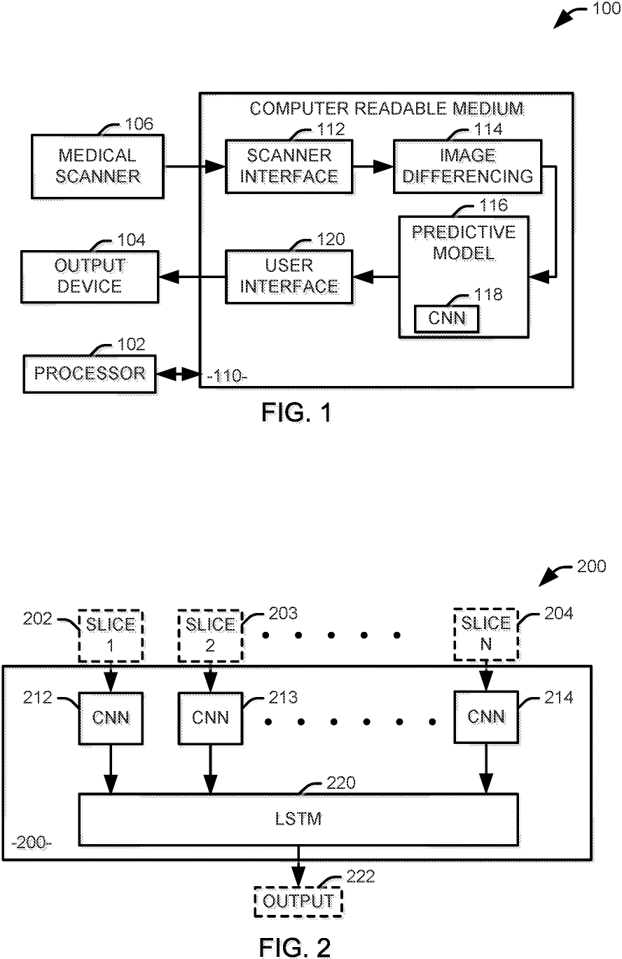

FIG. 1 illustrates a system 100 for evaluating of the stability of a joint in the foot and ankle complex via weight-bearing medical imaging in accordance with an aspect of the present invention. The system 100 includes a processor 102, an output device 104, a medical scanner 106, and a non-transitory computer readable medium 110 storing machine executable instructions that can be executed by the processor to perform various functions.

The machine executable instructions include, for example, an image interface 112 that is configured to receive a three-dimensional medical image of the joint, captured while the joint is bearing at least a portion of the subject’s weight, from the medical scanner 106, a remote system storing medical images, or a local storage medium. Depending on the source of the images, the image interface 112 can include, for example, software for interacting with appropriate hardware for implementing a bus or network connection with the source of the sequence of images.

A predictive model 114 includes at least a convolutional neural network 118 that receives the three-dimensional medical images as a sequence of two-dimensional slices and produces a parameter representing the stability of the joint. It will be appreciated that the parameter representing the stability of the joint can be categorical (e.g., “stable” or “not stable”) or continuous. Continuous parameters can include, for example, likelihoods that a given joint belongs in a particular category or a defined metric representing a stability of the joint. Categorical parameters can include clinical descriptors for the joint as well as ranges or likelihoods or stability metrics associated with the joint.

In one implementation, the convolutional neural network 118 provides the parameter representing the stability of the joint as a direct output. In another implementation, the convolutional neural network 118 is one of a plurality of independent convolutional neural networks that each receive one of the set of two-dimensional images and provide a parameter to an arbitrator to select a final parameter representing the stability of the joint via an arbitration scheme. In one example, an average (e.g., mean, median, or mode) of the parameters provided by the plurality of convolutional neural networks is provided as the final output. In another example, the plurality of convolutional neural networks each provide a continuous value, and an extremum of the values is selected as the final output. In still another example, the plurality of convolutional neural networks each provide a categorical or a continuous value, and the arbitrator can assign a final categorical parameter according to a set of logical rules. It will be appreciated that the implementation of the arbitrator can vary with the specific implementation, and that the examples provided herein are not exhaustive.

In still another implementation, one or more convolutional neural networks each receive one or more of the two-dimensional slices from the three-dimensional medical image and provides an array of values, for example, the output of a pooling layer of the convolutional neural network. These features can be provided to another machine learning algorithm that is part of the predictive model 114. In practice, the other machine learning algorithm can utilize additional parameters as part of assigning the parameter representing the stability of the joint, including, for example, biometric parameters (e.g., weight, height, blood pressure, blood glucose, etc.), and relevant medical history. It will be appreciated that the relevant additional parameters will vary with the implementation, and that the examples provided herein are not exhaustive.

The additional machine learning algorithm can utilize one or more pattern recognition algorithms, each of which analyze some or all of the features provided by the one or more convolutional neural networks to assign the parameter representing the stability of the joint. The training process of a given pattern recognition algorithm will vary with its implementation, but training generally involves a statistical aggregation of training data into one or more parameters associated with the output class or parameter. For rule-based models, such as decision trees, domain knowledge, for example, as provided by one or more human experts, can be used in place of or to supplement training data in selecting rules for classifying a user using the extracted features. Any of a variety of techniques can be utilized for the classification algorithm, including support vector machines, regression models, self-organized maps, fuzzy logic systems, data fusion processes, boosting and bagging methods, rule-based systems, or artificial neural networks.

For example, an SVM classifier can utilize a plurality of functions, referred to as hyperplanes, to conceptually divide boundaries in the N-dimensional feature space, where each of the N dimensions represents one associated feature of the feature vector. The boundaries define a range of feature values associated with each class. Accordingly, an output class and an associated confidence value can be determined for a given input feature vector according to its position in feature space relative to the boundaries. In one implementation, the SVM can be implemented via a kernel method using a linear or non-linear kernel.

An ANN classifier comprises a plurality of nodes having a plurality of interconnections. The values from the feature vector are provided to a plurality of input nodes. The input nodes each provide these input values to layers of one or more intermediate nodes. A given intermediate node receives one or more output values from previous nodes. The received values are weighted according to a series of weights established during the training of the classifier. An intermediate node translates its received values into a single output according to a transfer function at the node. For example, the intermediate node can sum the received values and subject the sum to a binary step function. A final layer of nodes provides the confidence values for the output classes of the ANN, with each node having an associated value representing a confidence for one of the associated output classes of the classifier.

Many ANN classifiers are fully-connected and feedforward. A convolutional neural network, however, includes convolutional layers in which nodes from a previous layer are only connected to a subset of the nodes in the convolutional layer. Recurrent neural networks are a class of neural networks in which connections between nodes form a directed graph along a temporal sequence. Unlike a feedforward network, recurrent neural networks can incorporate feedback from states caused by earlier inputs, such that an output of the recurrent neural network for a given input can be a function of not only the input but one or more previous inputs. As an example, Long Short-Term Memory (LSTM) networks are a modified version of recurrent neural networks, which makes it easier to remember past data in memory.

A rule-based classifier applies a set of logical rules to the extracted features to select an output class. Generally, the rules are applied in order, with the logical result at each step influencing the analysis at later steps. The specific rules and their sequence can be determined from any or all of training data, analogical reasoning from previous cases, or existing domain knowledge. One example of a rule-based classifier is a decision tree algorithm, in which the values of features in a feature set are compared to corresponding threshold in a hierarchical tree structure to select a class for the feature vector. A random forest classifier is a modification of the decision tree algorithm using a bootstrap aggregating, or “bagging” approach. In this approach, multiple decision trees are trained on random samples of the training set, and an average (e.g., mean, median, or mode) result across the plurality of decision trees is returned. For a classification task, the result from each tree would be categorical, and thus a modal outcome can be used. The clinical parameter can be displayed to a user at the output device 104 via the user interface 120. Additionally or alternatively, the clinical parameter can be stored in a memory, for example, in an electronic health records database, or used to assign the patient to a course of treatment. For example, where the joint is found to be unstable, the patient can be referred for physical therapy or surgical intervention.

In one implementation, the additional machine learning model is implemented as a recurrent neural network, such as a long, short-term memory network. A plurality of convolutional neural networks each provide the output of a pooling layer to the recurrent neural network as a set of features, and the recurrent neural network provides the clinical parameter based upon the received sets of features. FIG. 2 illustrates a predictive model 200 that can be used in a system for determining a clinical parameter via evaluation of sequential medical images. The predictive model 200 is configured to receive a sequence of medical images 202-204. Each of the sequence of medical images 202-204 is provided to one of a plurality of convolutional neural networks 212-214. Each convolutional neural network 212-214 is used to extract features from each input independently. One or more of the convolutional neural networks can be implemented as a pre-trained model, with the weights of some or all of the links within each convolutional neural network set using link weights from an existing model via transfer learning. The values of final pooling layer of each convolutional neural network 212-214 are provided as inputs to a long short-term memory (LSTM) network 220 that provides the clinical parameter.

Long short-term memory networks are a special kind of recurrent neural networks that are capable of selectively remembering patterns for long duration of time. The long-term memory is called the cell state, and the cell state is controlled by a set of gates including an input gate, an output gate, and a forget gate. A forget gate placed below the cell state is used to modify the cell states. The forget gate outputs values indicating which information to forget by multiplying zero to a position in the matrix. If the output of the forget gate is one, the information is kept in the cell. Input gates determine which information should enter the cell states. Finally, the output gate determines which information should be passed on to the next hidden state of the network. The output 222 of the long short-term memory network 220 can either be the clinical parameter or used to derive the clinical parameter as a function of the output 222. Where the clinical parameter is categorical, it can represent a binary classification stability for the joint (e.g., stable or unstable), degrees of stability, a change in the stability of the joint (e.g., increase, decrease, no change), a predicted stability of the joint after a period of time, or the presence or absence of a disorder affecting the stability of the joint. Where the clinical parameter is categorical, it can represent the degree of stability of the joint, a change in the degree of stability, a predicted stability of the joint after a period of time, or a likelihood associated with one of the categorical labels.

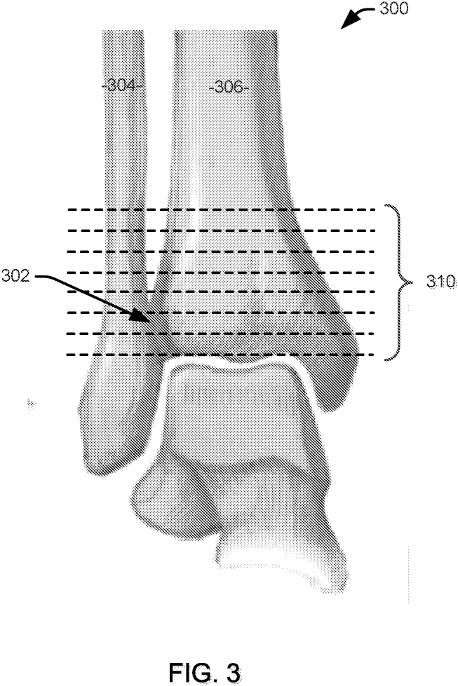

To demonstrate the benefits of the CNN-LSTM model of FIG. 2, the model was applied to the task of diagnosing tibiofibular syndesmosis instability. It will be appreciated that, while this example addresses syndesmosis instability, the model of FIG. 2 could be instead trained to recognize or identify other foot and ankle disease, injuries, or joint instability. FIG. 3 is a diagram 300 illustrating a portion of the foot and ankle complex that contains the syndesmosis joint 302, which is composed of the fibula 304 and tibia 306 bones. Injury to this joint 302 can lead to instability with excessive movement between the fibula 304 and tibia 306. Early recognition of syndesmosis instability is critical, but recognizing syndesmotic instability, especially when subtle, can be challenging. A variety of manually calculated measurements of the gap between the fibula and tibia bones (e.g. area, distance, volume measurements) have been shown to be useful metrics for distinguishing the injured and uninjured side, but such manual measurements performed by human readers can be time consuming, subject to variability and likely limited in diagnostic accuracy.

Accordingly, a computed tomography image of the joint can be captured while the ankle is bearing weight as a series of weight bearing computed tomography (WBCT) images or slices 310. The model was trained on retrospective data composed of WBCT images of stable and unstable tibiofibular syndesmosis including regions approximately five centimeters proximal to the tibial plafond. The dataset consisted of WBCT images for forty-eight patients with unstable joint, and ninety-six patients with stable joints. The “ground truth” labels (stable/unstable) were assigned to the images based on intraoperative confirmation of joint stability. The dataset was split into training, validation, and test subsets in 80:10:10 ratio. The training subset was used to train model parameters, the validation subset was used to optimize model hyperparameters, while the test subset was held-out and not presented to the model during training. The test subset was used only after model training was completed, to assess the performance of the model. The accuracy of the model was compared to that of the “ground truth” labels to quantify the performance of the model on the binary task of classifying a given CT image set as either a “stable” or “unstable” joint. The model was able to diagnose joint stability with 86.6% accuracy on the test subset.

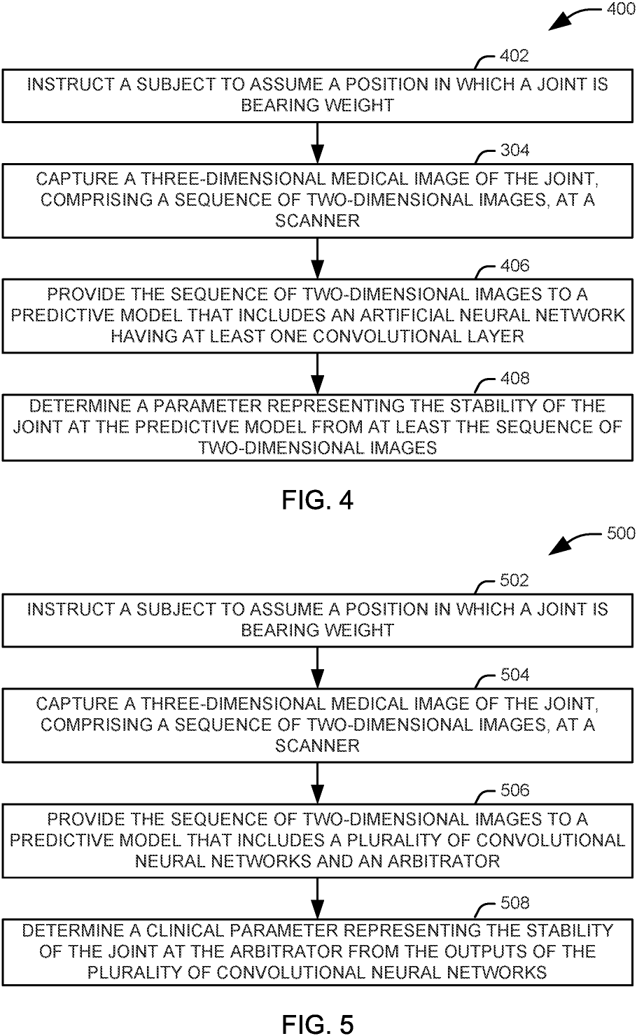

In view of the foregoing structural and functional features described above in FIGS. 1-3, example methods will be better appreciated with reference to FIGS. 4 and 5. While, for purposes of simplicity of explanation, the method of FIGS. 4 and 5 are shown and described as executing serially, it is to be understood and appreciated that the present invention is not limited by the illustrated order, as some actions could in other examples occur in different orders and/or concurrently from that shown and described herein.

FIG. 4 illustrates one example of a method 400 for evaluating a stability of a joint within the foot and ankle complex of a subject in accordance with an aspect of the present invention. In one example, the joint is the syndesmosis joint. At 402, a subject is instructed to assume a position in which the joint is bearing weight. It will be appreciated that the position can be selected to place varying amounts of weight on the joint, including a standing position in which the subject’s weight is distributed between both legs, a position in which all of the subject’s weight is supported through one leg, or a position in which at least a portion of the subject’s weight is supported by a mechanical support. At 404, a three-dimensional medical image of the joint, comprising a sequence of two-dimensional image slices, is captured at a scanner. It will be appreciated that the sequence of medical images can be stored in a computer readable medium between the capture of the images at 404 and the subsequent steps of the method. In one example, the three-dimensional medical image is captured from a computed tomography scanner or magnetic resonance imaging scanner.

At 406, the sequence of two-dimensional image slices is provided to a predictive model that includes an artificial neural network having at least one convolutional layer. The predictive model comprising an artificial neural network having at least one convolutional layer. In one implementation, the predictive model further includes another artificial neural network that receives an output of the artificial neural network having at least one convolutional layer and provides the parameter representing the stability of the joint. In one example, the artificial neural network can be implemented as a recurrent network, such as a long, short-term memory network. At 408, a parameter representing the stability of the joint is determined at the predictive model from at least the sequence of two-dimensional image slices. The parameter representing the stability of the joint can be categorical or continuous, and in one example, the parameter is categorical, with the predictive model labeling the joint as “stable” or “unstable”.

In one example, the predictive model comprises multiple independent convolutional neural networks, which each receive one of the set of two-dimensional image slices as an input. In this example, the output of each of the multiple convolutional neural networks is provided to either an arbitrator or another predictive algorithm to provide the clinical parameter. For example, each of the convolutional neural networks can provide their outputs as a set of features to an artificial neural network, such as a recurrent neural network, that determines the clinical parameter according to at least the provided set of features. The output of each convolutional neural network can be, for example, an array of values from a convolutional or pooling layer of the convolutional neural network.

FIG. 5 illustrates another method 500 for evaluating a stability of a joint within the foot and ankle complex of a subject in accordance with an aspect of the present invention. In one example, the joint is the syndesmosis joint. At 502, a subject is instructed to assume a position in which the joint is bearing weight. It will be appreciated that the position can be selected to place varying amounts of weight on the joint, including a standing position in which the subject’s weight is distributed between both legs, a position in which all of the subject’s weight is supported through one leg, or a position in which at least a portion of the subject’s weight is supported by a mechanical support. At 504, a three-dimensional medical image of the joint, comprising a sequence of two-dimensional image slices, is captured at a scanner. It will be appreciated that the sequence of medical images can be stored in a computer readable medium between the capture of the images at 504 and the subsequent steps of the method. In one example, the three-dimensional medical image is captured from a computed tomography scanner or magnetic resonance imaging scanner.

At 506, the sequence of two-dimensional image slices is provided to a predictive model comprising a plurality of convolutional neural networks and an arbitrator that receives the outputs of the plurality of convolutional neural networks. In one implementation, each of the plurality of convolutional neural networks receives, as an input, an image slice of the sequence of two-dimensional image slices, with each of the plurality of convolutional neural networks receiving a different image slice. The arbitrator can be implemented as any appropriate means for utilizing the outputs of the convolutional neural network for generating a continuous or categorical parameter representing the stability of the joint, from a voting system that aggregates categorical outputs from the convolutional neural networks to generate a final output to another machine learning model, such as a recurrent neural network, that uses the output of the convolutional neural networks as features for a classification or regression model. At 508, a clinical parameter representing the stability of the joint at the arbitrator from the outputs of the plurality of convolutional neural networks. The clinical parameter can be displayed to a user, stored on a computer readable medium, or employed by a user or automated system to assign the patient to a course of treatment.

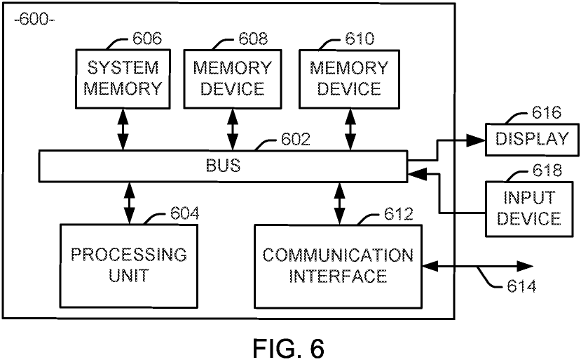

FIG. 6 is a schematic block diagram illustrating an exemplary system 600 of hardware components capable of implementing examples of the systems and methods disclosed herein. The system 600 can include various systems and subsystems. The system 600 can be a personal computer, a laptop computer, a workstation, a computer system, an appliance, an application-specific integrated circuit (ASIC), a server, a server BladeCenter, a server farm, etc.

The system 600 can include a system bus 602, a processing unit 604, a system memory 606, memory devices 608 and 610, a communication interface 612 (e.g., a network interface), a communication link 614, a display 616 (e.g., a video screen), and an input device 618 (e.g., a keyboard, touch screen, and/or a mouse). The system bus 602 can be in communication with the processing unit 604 and the system memory 606. The additional memory devices 608 and 610, such as a hard disk drive, server, standalone database, or other non-volatile memory, can also be in communication with the system bus 602. The system bus 602 interconnects the processing unit 604, the memory devices 606-610, the communication interface 612, the display 616, and the input device 618. In some examples, the system bus 602 also interconnects an additional port (not shown), such as a universal serial bus (USB) port.

The processing unit 604 can be a computing device and can include an application-specific integrated circuit (ASIC). The processing unit 604 executes a set of instructions to implement the operations of examples disclosed herein. The processing unit can include a processing core.

The additional memory devices 606, 608, and 610 can store data, programs, instructions, database queries in text or compiled form, and any other information that may be needed to operate a computer. The memories 606, 608 and 610 can be implemented as computer-readable media (integrated or removable), such as a memory card, disk drive, compact disk (CD), or server accessible over a network. In certain examples, the memories 606, 608 and 610 can comprise text, images, video, and/or audio, portions of which can be available in formats comprehensible to human beings.

Additionally or alternatively, the system 600 can access an external data source or query source through the communication interface 612, which can communicate with the system bus 602 and the communication link 614.

In operation, the system 600 can be used to implement one or more parts of a system for evaluating a stability of a joint within the foot and ankle complex of a subject in accordance with the present invention. Computer executable logic for implementing the system resides on one or more of the system memory 606, and the memory devices 608 and 610 in accordance with certain examples. The processing unit 604 executes one or more computer executable instructions originating from the system memory 606 and the memory devices 608 and 610. The term “computer readable medium” as used herein refers to a medium that participates in providing instructions to the processing unit 604 for execution. This medium may be distributed across multiple discrete assemblies all operatively connected to a common processor or set of related processors. Specific details are given in the above description to provide a thorough understanding of the embodiments. However, it is understood that the embodiments can be practiced without these specific details. For example, physical components can be shown in block diagrams in order not to obscure the embodiments in unnecessary detail. In other instances, well-known circuits, processes, algorithms, structures, and techniques can be shown without unnecessary detail in order to avoid obscuring the embodiments.

Implementation of the techniques, blocks, steps, and means described above can be done in various ways. For example, these techniques, blocks, steps, and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, microcontrollers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed, but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium” can represent one or more memories for storing data, including read only memory (ROM), random access memory (RAM), magnetic RAM, core memory, magnetic disk storage mediums, optical storage mediums, flash memory devices and/or other machine readable mediums for storing information. The term “machine-readable medium” includes, but is not limited to portable or fixed storage devices, optical storage devices, wireless channels, and/or various other storage mediums capable of storing that contain or carry instruction(s) and/or data.

What have been described above are examples. It is, of course, not possible to describe every conceivable combination of components or methodologies, but one of ordinary skill in the art will recognize that many further combinations and permutations are possible. Accordingly, the disclosure is intended to embrace all such alterations, modifications, and variations that fall within the scope of this application, including the appended claims. As used herein, the term “includes” means includes but not limited to, the term “including” means including but not limited to. The term “based on” means based at least in part on. Additionally, where the disclosure or claims recite “a,” “an,” “a first,” or “another” element, or the equivalent thereof, it should be interpreted to include one or more than one such element, neither requiring nor excluding two or more such elements.

What is claimed is:

1. A method for evaluating a stability of a joint within the foot and ankle complex of a subject comprising:
instructing a subject to assume a position in which the joint is bearing weight;
capturing a three-dimensional medical image of the joint, comprising a sequence of two-dimensional image slices, at a scanner;
providing the sequence of two-dimensional image slices to a predictive model, the predictive model comprising an artificial neural network having at least one convolutional layer; and
determining a clinical parameter representing the stability of the joint at the predictive model from at least the sequence of two-dimensional image slices.

2. The method of claim 1, wherein the predictive model comprises the artificial neural network having at least one convolutional layer and an machine learning model that receives an output of the artificial neural network having at least one convolutional layer and provides the clinical parameter representing the stability of the joint.

3. The method of claim 2, wherein the machine learning model is a recurrent neural network.

4. The method of claim 1, wherein the artificial neural network having at least one convolutional layer is a first convolutional neural network of a plurality of convolutional neural networks, each of the plurality of convolutional neural networks being independent of each other of the plurality of convolutional neural networks and providing an output to an arbitrator that provides the clinical parameter representing the stability of the joint.

5. The method of claim 4, wherein each of the plurality of convolutional neural networks receives, as an input, an image slice of the sequence of two-dimensional image slices, each of the plurality of convolutional neural networks receiving a different image slice of the sequence of two-dimensional image slices than each of the other of the plurality of convolutional neural networks.

6. The method of claim 4, wherein the arbitrator is a long short-term memory network.

7. The method of claim 1, wherein the joint is the syndesmosis joint.

8. The method of claim 1, further comprising assigning the patient to a course of treatment based upon the clinical parameter representing the stability of the joint.

9. A system comprising:
a medical scanner configured to capture images of a joint within the foot and ankle complex of a subject while the joint is bearing a weight of the subject to provide a sequence of at least three medical images;
a processor; and
a non-transitory computer readable medium storing executable instructions, the executable instructions comprising:
a scanner interface that receives the sequence of at least three medical images from the medical scanner; and
a predictive model, comprising an artificial neural network having at least one convolutional layer, that determines a clinical parameter representing a stability of the joint from at least the set of at least two difference images.

10. The system of claim 9, wherein the artificial neural network having at least one convolutional layer is a first convolutional neural network of a plurality of convolutional neural networks, each of the plurality of convolutional neural networks being independent of the other of the plurality of convolutional neural networks, and the predictive model further comprising an arbitrator that provides the clinical parameter from the outputs of the plurality of convolutional neural networks.

11. The system of claim 10, wherein the arbitrator comprises an artificial neural network, each of the plurality of convolutional neural networks providing an output as a set of features to the artificial neural network, the artificial neural network determining the clinical parameter according to at least the sets of features provided by the plurality of neural networks.

12. The system of claim 10, wherein each of the plurality of convolutional neural networks receives, as an input, a medical image of the sequence of at least three medical images, each of the plurality of convolutional neural networks receiving a different medical image than each of the other of the plurality of convolutional neural networks.

13. The system of claim 10, wherein the artificial neural network is a long short-term memory network.

14. The system of claim 9, wherein the scanner is a magnetic resonance imaging (MRI) system that provides the sequence of at least three medical images to the imager interface as a series of cross-sectional slices of a three-dimensional MRI image.

15. The system of claim 9, wherein the scanner is a computed tomography system (CT) that provides the sequence of at least three medical images to the imager interface as a series of cross-sectional slices of a three-dimensional CT image.

16. A method for evaluating a stability of a joint within the foot and ankle complex of a subject comprising:
instructing a subject to assume a position in which the joint is bearing weight;
capturing a three-dimensional medical image of the joint at a scanner comprising a sequence of two-dimensional image slices; and
providing the sequence of two-dimensional image slices to a predictive model, the predictive model comprising a plurality of convolutional neural networks and an arbitrator that receives the outputs of the plurality of convolutional neural networks; and
determining a clinical parameter representing the stability of the joint at the arbitrator from the outputs of the plurality of convolutional neural networks.

17. The method of claim 16, wherein each of the plurality of convolutional neural networks receives, as an input, an image slice of the sequence of two-dimensional image slices, each of the plurality of convolutional neural networks receiving a different image slice of the sequence of two-dimensional image slices than each of the other of the plurality of convolutional neural networks.

18. The method of claim 17, wherein the arbitrator is a recurrent neural network.

19. The method of claim 16, wherein the joint is the syndesmosis joint.

20. The method of claim 16, further comprising assigning the patient to a course of treatment based upon the clinical parameter representing the stability of the joint.
